(19) United States
(12) Patent Application Publication
(10) Pub. No.: US 2026/0306404 A1
(43) Pub. Date: Oct. 8, 2026

(54) LIPOSOME COMPOSITIONS AND MEDICAL USES THEREOF

(71) Applicant: CHOSA ONCOLOGY AB, Lund (SE)

(72) Inventors: Peter Buhl JENSEN, Lund (SE); Ulla BUHL, Lund (SE); Thomas JENSEN, Lund (SE); Steen KNUDSEN, Lund (SE); Anker HANSEN, Lund (SE); Mogens Winkel MADSEN, Lund (SE); Ulrik LASSEN, Lund (SE)

(21) Appl. No.: 19/481,670

(22) PCT Filed: May 8, 2024
(86) PCT No.: PCT/EP2024/062681

§ 371 Date: Nov. 5, 2025

(30) Foreign Application Priority Data
May 8, 2023 (EP) 23171983.2

(51) Int. Cl. A61K 9/1271; A61K 31/555; A61K 33/243; A61K 45/06; A61P 35/04

(52) U.S. Cl. CPC A61K 9/1271; A61K 31/555; A61K 33/243; A61K 45/06; A61P 35/04

(57) ABSTRACT

The present disclosure describes liposomes comprising an anti-cancer agent, for example cisplatin and their use in treatment of cancer patients, for example subgroups of cancer patients.

### TECHNICAL FIELD

The present disclosure describes liposomes comprising an anti-cancer agent, for example cisplatin or carboplatin and their use in treatment of cancer patients, for example subgroups of cancer patients.

### BACKGROUND

Liposomes are microscopic spheres which were developed as drug delivery vehicles/systems in the 1980s. The first liposome-based pharmaceuticals were approved for commercial use in the 1990s. Repeated dosing of PEGylated liposomal formulations has in some cases resulted in an Accelerated Blood Clearance (ABC-phenomenon) leading to a fast clearance from the bloodstream and corresponding increased accumulation in liver and spleen when compared to the first dose. The ABC-phenomenon may cause unintended release of encapsulated compound in organs having accumulated liposomes. Moreover, the ABC-phenomenon is typically non-desired as it may prevent the liposomes from accumulating at intended sites. Various targeting strategies for liposomes have been described, e.g. conjugation to cell specific ligands such as antibodies.

Anti-cancer agents are known to have serious side effects, and the most important side effects of the platinum based agent, cisplatin in its free form are listed below:
Nephrotoxicity—The major dose-limiting toxicity of cisplatin is dose-related and cumulative renal insufficiency. The administration of cis-platin using a 6- to 8-hour infusion with intravenous hydration has been used to reduce nephrotoxicity. However, renal toxicity still can occur after utilization of these procedures.
Ototoxicity—Ototoxicity has been observed in up to 31% of patients treated with a single dose of cisplatin 50 mg/m², and is manifested by tinnitus and/or hearing loss in the high frequency range (4,000 to 8,000 Hz). Ototoxic effects may be related to the peak plasma concentration of cisplatin.
Hematologic—Myelosuppression occurs in 25% to 30% of patients treated with cisplatin. Leukopenia and thrombocytopenia are more pronounced at higher doses (>50 mg/m²). Anemia occurs at approximately the same frequency as leukopenia and thrombocytopenia.
Gastrointestinal—Marked nausea and vomiting occur in almost all patients treated with cisplatin, and are occasionally so severe that the drug must be discontinued. Nausea and vomiting usually begin within 1 to 4 hours after treatment and last up to 24 hours.
Serum Electrolyte Disturbances—Hypomagnesemia, hypocalcemia, hyponatremia, hypokalemia, and hypophosphatemia have been reported to occur in patients treated with cisplatin and are probably related to renal tubular damage.
Neurotoxicity—Neurotoxicity is usually characterized by peripheral neuropathies. The neuropathies usually occur after prolonged therapy (4 to 7 months); however, neurologic symptoms have been reported to occur after a single dose.
Hepatotoxicity—Transient elevations of liver enzymes, especially SGOT, as well as bilirubin, have been reported to be associated with cisplatin administration at the recommended doses.

Treatment using anti-cancer agents, and in particular free cisplatin has a number of potential side effects and there is a need for cisplatin formulations with a reduced risk of side effects. Furthermore, there is a need for providing personalized treatments, where patients likely to respond well to the treatment can be identified prior to treatment.

### SUMMARY

Over this background art the present inventors have now found liposomes comprising an anti-cancer agent and pharmaceutical compositions comprising such liposomes that can be used in the treatment of cancer, in particular to reduce selected side effects associated with the anti-cancer agent. Furthermore, the present inventors have implemented a screening method (DRP) in the clinical setting for identifying patients that have increased likelihood of responding well to the liposomes of the present disclosure.

Accordingly, in a first aspect the disclosure provides a liposome comprising:
a) an anti-cancer agent, for example cisplatin, and
b) one or more amphiphilic molecules.

In a further aspect, the disclosure provides a plurality of liposomes as defined herein, wherein the plurality of the liposomes is characterized by a diameter of from 50 to 400 nm, such as from 80 to 160 nm, for example 90 to 120 nm.

In a further aspect, the disclosure provides a plurality of liposomes as defined herein, wherein the plurality of the liposomes is characterized by a polydispersity index (PDI) of 0.2 or less, such as 0.10 or less.

In a further aspect, the disclosure provides a pharmaceutical composition comprising the liposome as defined herein.

In a further aspect, the disclosure provides a liposome or a pharmaceutical composition as defined herein for use in the treatment of a cancer in a patient.

In a further aspect, the disclosure provides a liposome for use in the treatment of breast cancer, such as metastatic breast cancer in a patient having a cisplatin drug response predictor (DRP) score of at least 80%, comprising administering a liposome comprising:
a) 1,2-distearoyl-sn-glycero-3-[phospho-rac-(1-glycerol)] (DSPG);
b) 1,2-distearovl-sn-glycero-3-phosphoethanolamine-N-[methoxy (polyethylene glycol)-2000)] (DSPE-PEG);
c) 1,2-distearoyl-sn-glycero-3-phosphocholine (DSPC); at a mol ratio of: 25:5:70 of a), b) to c),
d) less than 1% cholesterol, and

cisplatin, to the patient.

### INCORPORATION BY REFERENCE

All publications, patents, and patent applications referred to herein are incorporated by reference to the same extent as if each individual publication, patent, or patent application was specifically and individually indicated to be incorporated by reference. In the event of a conflict between a term herein and a term in an incorporated reference, the term herein prevails and controls.

### DETAILED DESCRIPTION

The features and advantages of the present invention is readily apparent to a person skilled in the art by the below detailed description of embodiments and examples of the invention with reference to the figures and drawings included herein.

### Liposome

In some embodiments, the present disclosure provides a liposome comprising:
a) an anti-cancer agent, for example cisplatin, and
b) one or more amphiphilic molecules.

In some embodiments, the liposome provided is of pharmaceutical grade.

The liposome of the present disclosure is in some embodiments hydrolysable under physiological conditions, for example in cancerous tissue.

Preferably, in some embodiments the liposome comprises:
a) 1,2-distearoyl-sn-glycero-3-[phospho-rac-(1-glycerol)] (DSPG);
b) 1,2-distearovl-sn-glycero-3-phosphoethanolamine-N-[methoxy (polyethylene glycol)-2000)] (DSPE-PEG);
c) 1,2-distearoyl-sn-glycero-3-phosphocholine (DSPC); at a mol ratio of: 25:5:70 of a), b) to c),
d) less than 1% cholesterol, and
e) cisplatin.

In some embodiments, the liposome is provided wherein:
a) an interior of the liposome comprises 0.9% NaCl;
b) a buffer solution on an exterior of the liposome comprises 10 mM phosphate buffer at pH 6.5, 1 mM NaCl and 10% sucrose; and/or
c) the liposome is unilamellar.

In some embodiments, a plurality of liposomes as defined in any one of the preceding claims, wherein the plurality of the liposomes is characterized by a diameter of from 50 to 400 nm, such as from 80 to 160 nm, for example 90 to 120 nm.

In some embodiments, the plurality of the liposomes is characterized by a polydispersity index (PDI) of 0.2 or less, such as 0.10 or less.

### Liposome Effects

In some embodiments, the use of the liposome of the present disclosure reduces at least one adverse effect resulting from treatment with the anti-cancer agent. In some embodiments, the at least one adverse effect resulting from treatment with the anti-cancer agent is a disorder with hearing impairment.

In some embodiments, the at least one adverse effect resulting from treatment with the anti-cancer agent is selected from the group consisting of: ototoxic hearing loss, sensorineural hearing loss, auditory neuropathy spectrum disorder (ANSD), tinnitus, and neurotoxicity, such as peripheral neurotoxicity.

In some embodiments, the adverse effects resulting from treatment with the anti-cancer agent is selected from the group consisting of: ototoxic hearing loss and peripheral neurotoxicity.

In some embodiments, the adverse effects resulting from treatment with the anti-cancer agent is selected from the group consisting of: loss of vibration sense, paraesthesia, and sensory ataxia.

### Anti-Cancer Agents

In some embodiments, the present disclosure provides an anti-cancer which is a platinum-based anti-cancer agent. In some embodiments, the anti-cancer agent is an ototoxic platinum-based anti-cancer agent.

In some embodiments, the anti-cancer agent is a platinum-based anti-cancer agent selected from the group consisting of: cisplatin and carboplatin.

### Amphiphilic Molecules

In some embodiments, the one or more amphiphilic molecules are capable of or suitable for assembling into a liposome.

In some embodiments, the one or more amphiphilic molecules are selected from the group consisting of: phospholipids, glycolipids, steroids, fatty acids, fatty sulphates, amphiphilic peptides, polyesters, polyamides, polyurethanes, poly(lactic-co-glycolic acid) (PLGA) polymers, and co-block polymers comprising PEG.

In some embodiments, the liposome comprises a plurality of different amphiphilic molecules.

In some embodiments, the plurality of different amphiphilic molecules comprises an anionic lipid, a neutral lipid, and a polymer conjugated lipid.

In some embodiments, the liposome comprises between 20% and 45% of an anionic lipid.

In some embodiments, the liposome comprises between 40% and 75% of a neutral lipid.

In some embodiments, the liposome comprises between 3% and 6% of a polymer conjugated lipid.

In some embodiments, the anionic lipid comprises a phospholipid selected from the group consisting of PI (phosphatidyl inositol), PS (phosphatidyl serine), DPG (bisphosphatidyl glycerol), PA (phosphatidic acid), PEOH (phosphatidyl alcohol), and PG (phosphatidyl glycerol).

In some embodiments, the anionic lipid is a phospholipid selected from the group consisting of DSPI (distearoyl phosphatidyl inositol), DSPS (distearoyl phosphatidyl serine), DSDPG (distearoyl bisphosphatidyl glycerol), DSPA (distearoyl phosphatidic acid), DSPEOH (distearoyl phosphatidyl alcohol), and DSPG (distearoyl phosphatidyl glycerol), wherein the lipid comprises two stearoyl chains.

In some embodiments, the neutral lipid is distearoylphosphatidylcholine (DSPC). In some embodiments, the polymer conjugated lipid is DSPE-PEG, such as DSPE-PEG-2000.

In some embodiments, the liposome comprises a polymer conjugated lipid, wherein the polymer is PEG and the PEG is selected from PEG-500 to PEG-5000, such as from PEG-1000 to PEG-4000, such as from PEG-1500 to PEG-3500, for example PEG-2000.

In some embodiments, the liposome comprises an amphphilic molecule selected from the group consisting of: PEG [poly(ethylene glycol)], PAcM [poly(N-acryloylmorpholine)], PVP [poly(vinylpyrrolidone)], PLA [poly(lactide)], PG [poly(glycolide)], POZO [poly(2-methyl-2-oxazoline)], PVA [poly(vinyl alcohol)], HPMC (hydroxypropylmethylcellulose), PEO [poly(ethylene oxide)], chitosan [poly(D-glucosamine)], PAA [poly(aminoacid)], polyHEMA [Poly(2-hydroxyethylmethacrylate)] and co-polymers thereof.

Pharmaceutical Composition with Liposome

In some embodiments, a pharmaceutical composition is provided comprising the liposome of the present disclosure. In some embodiments, the pharmaceutical composition further comprises one or more excipients, carriers and/or adjuvants.

### Treatment of Cancer

In some embodiments, a liposome or a pharmaceutical composition is provided for use in the treatment of a cancer in a patient. Thus, the present disclosure provides a method for treatment of a cancer in a patient using a liposome or pharmaceutical composition as defined herein.

In some embodiments, the cancer is being treated using a liposome comprising:
a) 1,2-distearoyl-sn-glycero-3-[phospho-rac-(1-glycerol)] (DSPG);
b) 1,2-distearovl-sn-glycero-3-phosphoethanolamine-N-[methoxy (polyethylene glycol)-2000)] (DSPE-PEG);
c) 1,2-distearoyl-sn-glycero-3-phosphocholine (DSPC); at a mol ratio of: 25:5:70 of a), b) to c),
d) less than 1% cholesterol, and
e) cisplatin.

Of particular relevance is the provision by the present disclosure of a liposome for use in treatment of breast cancer, such as metastatic breast cancer in a patient having a DRP (DRP is specified under “patients and prediction model”) of at least 80%. In some preferred embodiments, a method is provided for treatment of breast cancer, such as metastatic breast cancer in a patient having a DRP of at least 80%, wherein the liposome is as defined in of the present disclosure.

### Cancers

In some embodiments, the cancer is sensitive to treatment by the anti-cancer agent, such as cisplatin or carboplatin, for example cisplatin.

In some embodiments, the cancer sensitive to treatment by the anti-cancer agent is selected from the group consisting of: testicular cancer, ovarian cancer, bladder cancer, lung cancer, head and neck cancer (including throat cancer and nasopharyngeal cancer), cervical cancer, esophageal cancer, stomach cancer, colorectal cancer, and pancreatic cancer. In some embodiments, the cancer is breast cancer.

In some embodiments, the cancer is selected from the group consisting of: lung cancer, head and neck cancer, bladder cancer, pancreas cancer, lymphoma, skin cancer, cholangiocarcinoma, breast cancer, testicular cancer, and ovarian cancer. In the context where there anti-cancer agent is cisplatin or carboplatin, these cancers are particularly relevant as they are known to respond to treatment with cisplatin and/or carboplatin. In some embodiments, the cancer is lung cancer.

In particular embodiments, the cancer is metastatic breast cancer. Treatment of metastatic breast cancer using liposomes of the present disclosure is demonstrated by the present example 1.

In some embodiments, the cancers treated are those more commonly found among pediatric patients. In some embodiments, the cancer is selected from the group consisting of: osteosarcoma, neuroblastoma, Wilms tumor, rhabdomyosarcoma, germ cell tumors, and hepatoblastoma.

### Patients and Prediction Model

The present disclosure relates to treatment of cancer patients. In some embodiments, patients are selected such that the liposome of the present disclosure is expected to be particular beneficial with respect to a positive outcome of the treatment. In some embodiments, patients are selected based on a “cisplatin drug response predictor” (DRP) model demonstrated in Buhl IK, Santoni-Rugiu E, Ravn J, Hansen A, Christensen I J, Jensen T, et al. (2018) Molecular prediction of adjuvant cisplatin efficacy in Non-Small Cell Lung Cancer (NSCLC)—validation in two independent cohorts. PLoS ONE 13 (3). In some embodiments, the DRP model used to generate the “DRP score” is as demonstrated in U.S. Pat. No. 10,907,214, EP 3342879, WO 2008/138578, U.S. Pat. Nos. 8,445,198, 9,598,734, EP 2563936 or WO 2011/135459. The specific steps disclosed in these patent documents are fully incorporated herein to assist the skilled person in determining the DRP score of a subject. The prediction model and its methodology is fully incorporated herein.

Generally, the DRP score is determined using the following steps:
a) analyzing a biopsy from a cancer subject, such as a subject as defined herein;
b) applying an algorithm to expression state of 205 genes to obtain score;
c) devise treatment decision based on score, wherein a score of 80% or above is indicative of benefit from a treatment of the present disclosure, and
d) optionally providing the treatment to the subject having a score of 80% or above.

In some embodiments, the patient has a cisplatin drug response predictor (DRP) score of at least 80%. As shown in Example 1, a cut-off DRP score exist which can be used to evaluate whether a patient will likely respond well to the treatment. In some embodiments, this cut-off DRP score is 80%, such that patients having a DRP score of 80% or higher, such as from 80% to 100%, such as 85% or higher, such as 90% or higher will respond well to the treatment of the present disclosure.

In some embodiments, the patient has a cisplatin drug response predictor (DRP) score of at least 80% and the cancer is breast cancer, such as metastatic breast cancer.

In some embodiments, the patient is selected from the group consisting of: a newly diagnosed cancer patient, a survivor (patient having completed cancer treatment and is considered to be in remission or have no evidence of disease), a relapsed or refractory cancer patient, a palliative care cancer patient, a long-term follow-up cancer patient, and a supportive care cancer patient.

In some embodiments, the patient is a pediatric patient. Administering the liposome comprising a platinum-based anti-cancer agent, such as cisplatin or carboplatin to a cancer patient reduces the ototoxicity of the anti-cancer agent. This is particularly important for treatment of pediatric patients.

In some embodiments, the patient is selected from the group consisting of: a neonate (from birth through the first 28 days of life), an infant (29 days to less than 2 years), a child (2 years to less than 12 years), and an adolescent (aged 12 to but not including 22 years).

In some embodiments, the patient has one or more disorders selected from the group consisting of: ototoxic hearing loss, sensorineural hearing loss, auditory neuropathy spectrum disorder (ANSD), tinnitus, and neurotoxicity, such as peripheral neurotoxicity.

In some embodiments, the patient is at increased risk of developing one or more disorders selected from the group consisting of: ototoxic hearing loss, sensorineural hearing loss, auditory neuropathy spectrum disorder (ANSD), tinnitus, and neurotoxicity, such as peripheral neurotoxicity.

### Treatment Regimen

In some embodiments, the liposome or pharmaceutical composition is administered to the patient once a week. In some embodiments, the liposome or pharmaceutical composition is administered to the patient in one or more treatment cycles, wherein each treatment cycle comprises dosing on day 1 and day 8.

In some embodiments, the liposome or pharmaceutical composition is administered to the patient in one or more treatment cycles, wherein each treatment cycle comprises dosing on day 1 and day 8 every two, three, or four weeks, for example every three weeks.

In some embodiments, the liposome or pharmaceutical composition is administered to the patient in one or more treatment cycles, wherein each treatment cycle comprises dosing on day 1 and day 8 every three or four weeks.

In some embodiments, the liposome or pharmaceutical composition is administered to the patient over from 2 to 8 treatment cycles, such as from 2 to 7 treatment cycles, for example from 3 to 6, such as from 4 to 5 treatment cycles.

In some embodiments, the method/use disclosed herein provides for (in the patient) from 80 mg to 120 mg cisplatin per treatment cycle, for example 75 mg.

In some embodiments, each dose of the anti-cancer agent administered to the patient is from 20 to 60 mg/m², such as from 30 to 50 mg/m², for example 40 mg/m². In some embodiments, these doses are particular relevant when the anti-cancer agent is cisplatin or carboplatin, preferably cisplatin.

### Combination Treatment

In some embodiments, the liposome or pharmaceutical composition is administered to the patient in combination with one or more further anti-cancer agents.

### Examples

### Materials and Methods

Chemicals used in the examples herein, e.g. for buffers and substrates, are commercial products of at least reagent grade.

Example 1—Predictive biomarker for cisplatin in prospective phase 2 of liposomal cisplatin in metastatic breast cancer.

### Materials—Preparation of LiPlaCis

A lipid intermediate is prepared by spray-drying the following a mixture of phospholipids (70/25/5 mol % DSPC/DSPG/DSPE-PEG2000). The lipids are dissolved in methanol and chloroform. The lipid intermediate is hydrated in an aqueous solution of the anti-cancer drug with agitation. At this step the liposomes are formed but they have a broad size distribution and is a mixture of single-layer and multiple-layer liposomes. In order to get a product with a narrow size distribution and mono-layer liposomes the hydration mixture is extruded by passing it through poly-carbonate filters of appropriate pore sizes. To remove un-encapsulated anti-cancer drug the mixture is purified. A number of techniques are available e.g. dialysis, gel-filtration and ultra-filtration. For preparations ranging from a few liters and above ultra-filtration is the preferred method. Preparations intended for parenteral administration must be sterilized e.g. by sterile-filtration.

### Methods

The dose-escalation part of a phase 1/2 study, of 20 patients with a variety of locally advanced or metastatic solid tumors, suggested a phase 2 dose of LiPlaCis to be 75 mg on day 1 and 8 q 3 wks. In the phase 2 part, the cisplatin drug response predictor (DRP) was used to select metastatic breast cancer (mBC) patients with response likelihood scores from FFPE diagnostic or later biopsies. The “cisplatin drug response predictor” (DRP) model was demonstrated in Buhl IK, Santoni-Rugiu E, Ravn J, Hansen A, Christensen I J, Jensen T, et al. (2018) Molecular prediction of adjuvant cisplatin efficacy in Non-Small Cell Lung Cancer (NSCLC)—validation in two independent cohorts. PLoS ONE 13 (3). Patients were treated until progression or unacceptable toxicity. Halfway through the phase 2 part, the dosing was changed to a per body surface schedule (40 mg/m²) after a fatal event with the flat dosing (in effect 51 mg/m² for this patient). Among the 52 patients included in the phase 2 part, data was reported from the 37 patients with mBC, being platinum naïve, with DRP scores 34-100%, and with RECIST-defined response outcomes. Within this subgroup, a post hoc analysis was conducted of possible DRP cut-off values that could identify patients with a clinically meaningful response to treatment.

### Results

mBC patients had received a median of 7 previous treatment lines. A DRP score ≥80% (DRP80+) discriminated well between responders and non-responders to liposomal cisplatin. All 4 partial remissions in the study were in the DRP80+group, and other key efficacy endpoints were in favor of the DRP80+vs the lower scores (DRP80-).

| | | Total | | DRP80− | | DRP80+ | | p-value² |
|---|---|---|---|---|---|---|---|---|
| N | | 37 | | 21 | | 16 | | |
| Tumor response | ORR | 4 | (10.8%) | 0 | (0%) | 4 | (25.0%) | 0.0276 |
| | CBR | 8 | (21.6%) | 2 | (9.5%) | 6 | (37.5%) | 0.0554 |
| PFS (in weeks) | median [95% CI] | 15 | [7, 24] | 8 | [6, 23] | 19 | [13, 30] | 0.155 |
| OS (in weeks) | median [95% CI] | 50 | [33, 60] | 44 | [21, 60] | 56 | [17, 62] | 0.554 |

²DRP80+ vs DRP80−

LiPlaCis was generally well tolerated, with a mean of 4.4 treatment cycles administered per patient. Only 1 SAE (pyelonephritis) was reported and considered related to treatment. One patient died whilst on treatment, but due to disease progression. From a total of 164 treatment cycles, 18 of the patients reported 1 grade 4 and 41 grade 3 AEs considered possibly related to treatment.

### CONCLUSIONS

LiPlaCis shows efficacy in heavily pretreated mBC patients, especially when using the DRP method as a companion diagnostic. Further, this example supports that patients having a DRP score of at least 80% respond surprisingly well to treatment by the liposome of the present disclosure.

1. A liposome comprising:
a) an anti-cancer agent, for example cisplatin, and
b) one or more amphiphilic molecules.

2. The liposome according to claim 1, wherein the anti-cancer agent is a platinum-based anti-cancer agent.

3. The liposome according to any one of the preceding claims, wherein the anti-cancer agent is an ototoxic platinum-based anti-cancer agent.

4. The liposome according to any one of the preceding claims, wherein the anti-cancer agent is a platinum-based anti-cancer agent selected from the group consisting of: cisplatin and carboplatin.

5. The liposome according to any one of the preceding claims, wherein the one or more amphiphilic molecules are capable of or suitable for assembling into a liposome.

6. The liposome according to any one of the preceding claims, wherein the liposome is of pharmaceutical grade.

7. The liposome according to any one of the preceding claims, wherein the one or more amphiphilic molecules are selected from the group consisting of: phospholipids, glycolipids, steroids, fatty acids, fatty sulphates, amphiphilic peptides, polyesters, polyamides, polyurethanes, poly(lactic-co-glycolic acid) (PLGA) polymers, and co-block polymers comprising PEG.

8. The liposome according to any one of the preceding claims, wherein the liposome comprises a plurality of different amphiphilic molecules.

9. The liposome according to any one of the preceding claims, wherein the plurality of different amphiphilic molecules comprises an anionic lipid, a neutral lipid, and a polymer conjugated lipid.

10. The liposome according to any one of the preceding claims, wherein the liposome comprises between 20% and 45% of an anionic lipid.

11. The liposome according to any one of the preceding claims, wherein the liposome comprises between 40% and 75% of a neutral lipid.

12. The liposome according to any one of the preceding claims, wherein the liposome comprises between 3% and 6% of a polymer conjugated lipid.

13. The liposome according to any one of the preceding claims, wherein the anionic lipid comprises a phospholipid selected from the group consisting of PI (phosphatidyl inositol), PS (phosphatidyl serine), DPG (bisphosphatidyl glycerol), PA (phosphatidic acid), PEOH (phosphatidyl alcohol), and PG (phosphatidyl glycerol).

14. The liposome according to any one of the preceding claims, wherein the anionic lipid is a phospholipid selected from the group consisting of DSPI (distearoyl phosphatidyl inositol), DSPS (distearoyl phosphatidyl serine), DSDPG (distearoyl bisphosphatidyl glycerol), DSPA (distearoyl phosphatidic acid), DSPEOH (distearoyl phosphatidyl alcohol), and DSPG (distearoyl phosphatidyl glycerol), wherein the lipid comprises two stearoyl chains.

15. The liposome according to any one of the preceding claims, wherein the neutral lipid is distearoylphosphatidylcholine (DSPC).

16. The liposome according to any one of the preceding claims, wherein the polymer conjugated lipid is DSPE-PEG, such as DSPE-PEG-2000.

17. The liposome according to any one of the preceding claims, wherein the liposome comprises a polymer conjugated lipid, wherein the polymer is PEG and the PEG is selected from PEG-500 to PEG-5000, such as from PEG-1000 to PEG-4000, such as from PEG-1500 to PEG-3500, for example PEG-2000.

18. The liposome according to any one of the preceding claims, wherein the liposome comprises an amphiphilic molecule selected from the group consisting of: PEG [poly(ethylene glycol)], PAcM [poly(N-acryloylmorpholine)], PVP [poly(vinylpyrrolidone)], PLA [poly(lactide)], PG [poly(glycolide)], POZO [poly(2-methyl-2-oxazoline)], PVA [poly(vinyl alcohol)], HPMC (hydroxypropylmethylcellulose), PEO [poly(ethylene oxide)], chitosan [poly(D-glucosamine)], PAA [poly(aminoacid)], polyHEMA [Poly(2-hydroxyethylmethacrylate)] and co-polymers thereof.

19. The liposome according to any one of the preceding claims, wherein the liposome is hydrolysable under physiological conditions, for example in cancerous tissue.

20. The liposome according to any one of the preceding claims, wherein the liposome comprises:
a) 1,2-distearoyl-sn-glycero-3-[phospho-rac-(1-glycerol)] (DSPG);
b) 1,2-distearovl-sn-glycero-3-phosphoethanolamine-N-[methoxy (polyethylene glycol)-2000)] (DSPE-PEG);
c) 1,2-distearoyl-sn-glycero-3-phosphocholine (DSPC);
at a mol ratio of: 25:5:70 of a), b) to c),
d) less than 1% cholesterol, and
e) cisplatin.

21. The liposome according to any one of the preceding claims, wherein
a) an interior of the liposome comprises 0.9% NaCl;
b) a buffer solution on an exterior of the liposome comprises 10 mM phosphate buffer at pH 6.5, 1 mM NaCl and 10% sucrose; and/or
c) the liposome is unilamellar.

22. A plurality of liposomes as defined in any one of the preceding claims, wherein the plurality of the liposomes is characterized by a diameter of from 50 to 400 nm, such as from 80 to 160 nm, for example 90 to 120 nm.

23. A plurality of liposomes as defined in any one of the preceding claims, wherein the plurality of the liposomes is characterized by a polydispersity index (PDI) of 0.2 or less, such as 0.10 or less.

24. A pharmaceutical composition comprising the liposome as defined according to any one of the preceding claims.

25. The pharmaceutical composition according to claim 24, wherein the pharmaceutical composition further comprises one or more excipients, carriers and/or adjuvants.

26. A liposome or a pharmaceutical composition as defined in any one of the preceding claims, for use in the treatment of a cancer in a patient.

27. The liposome or pharmaceutical composition for use according to claim 26, wherein the cancer is sensitive to treatment by the anti-cancer agent, such as cisplatin or carboplatin, for example cisplatin.

28. The liposome or pharmaceutical composition for use according to claim 27, wherein the cancer sensitive to treatment by the anti-cancer agent is selected from the group consisting of: testicular cancer, ovarian cancer, bladder cancer, lung cancer, head and neck cancer (including throat cancer and nasopharyngeal cancer), cervical cancer, esophageal cancer, stomach cancer, colorectal cancer, and pancreatic cancer.

29. The liposome or pharmaceutical composition for use according to claims 26-27, wherein the cancer is selected from the group consisting of: lung cancer, head and neck cancer, bladder cancer, pancreas cancer, lymphoma, skin cancer, cholangiocarcinoma, breast cancer, testicular cancer, and ovarian cancer.

30. The liposome or pharmaceutical composition for use according to claims 26-29, wherein the cancer is breast cancer.

31. The liposome or pharmaceutical composition for use according to claims 26-29, wherein the cancer is lung cancer.

32. The liposome or pharmaceutical composition for use according to claims 26-29, wherein the cancer is metastatic breast cancer.

33. The liposome or pharmaceutical composition for use according to claim 26, wherein the cancer is selected from the group 20 of: osteosarcoma, neuroblastoma, Wilms tumor, rhabdomyosarcoma, germ cell tumors, and hepatoblastoma.

34. The liposome or pharmaceutical composition for use according to claims 26-33, wherein the patient has a cisplatin drug response predictor (DRP) score of at least 80%.

35. The liposome or pharmaceutical composition for use according to claims 26-33, wherein the patient has a cisplatin drug response predictor (DRP) score of at least 80% and wherein the cancer is breast cancer, such as metastatic breast cancer.

36. The liposome or pharmaceutical composition for use according to claims 26-33, wherein the patient has a cisplatin drug response predictor (DRP) score of at least 80% and wherein the cancer is lung cancer.

37. The liposome or pharmaceutical composition for use according to claims 26-34, wherein the patient is selected from the group consisting of: a newly diagnosed cancer patient, a survivor (patient having completed cancer treatment and is considered to be in remission or have no evidence of disease), a relapsed or refractory cancer patient, a palliative care cancer patient, a long-term follow-up cancer patient, and a supportive care cancer patient.

38. The liposome or pharmaceutical composition for use according to claims 26-37, wherein the patient is a pediatric patient.

39. The liposome or pharmaceutical composition for use according to claims 26-38, wherein the patient is selected from the group consisting of: a neonate (from birth through the first 28 days of life), an infant (29 days to less than 2 years), a child (2 years to less than 12 years), and an adolescent (aged 12 to but not including 22 years).

40. The liposome or pharmaceutical composition for use according to claims 26-39, wherein the patient has one or more disorders selected from the group consisting of: ototoxic hearing loss, sensorineural hearing loss, auditory neuropathy spectrum disorder (ANSD), tinnitus, and neurotoxicity, such as peripheral neurotoxicity.

41. The liposome or pharmaceutical composition for use according to claims 26-39, wherein the patient is at increased risk of developing one or more disorders selected from the group consisting of: ototoxic hearing loss, sensorineural hearing loss, auditory neuropathy spectrum disorder (ANSD), tinnitus, and neurotoxicity, such as peripheral neurotoxicity.

42. The liposome or pharmaceutical composition for use according to claims 26-39, wherein the use reduces at least one adverse effect resulting from treatment with the anti-cancer agent.

43. The liposome or pharmaceutical composition for use according to claim 42, wherein the at least one adverse effect resulting from treatment with the anti-cancer agent is a disorder with hearing impairment.

44. The liposome or pharmaceutical composition for use according to claim 43, wherein the at least one adverse effect resulting from treatment with the anti-cancer agent is selected from the group consisting of: ototoxic hearing loss, sensorineural hearing loss, auditory neuropathy spectrum disorder (ANSD), tinnitus, and neurotoxicity, such as peripheral neurotoxicity.

45. The liposome or pharmaceutical composition for use according to claim 44, wherein the adverse effects resulting from treatment with the anti-cancer agent is selected from the group consisting of: ototoxic hearing loss and peripheral neurotoxicity.

46. The liposome or pharmaceutical composition for use according to claims 42-45, wherein the adverse effects resulting from treatment with the anti-cancer agent is selected from the group consisting of: loss of vibration sense, paraesthesia, and sensory ataxia.

47. The liposome or pharmaceutical composition for use according to any one of claims 26-46, wherein the liposome or pharmaceutical composition is administered to the patient once a week.

48. The liposome or pharmaceutical composition for use according to any one of claims 26-47, wherein the liposome or pharmaceutical composition is administered to the patient in one or more treatment cycles, wherein each treatment cycle comprises dosing on day 1 and day 8.

49. The liposome or pharmaceutical composition for use according to any one of claims 26-48, wherein the liposome or pharmaceutical composition is administered to the patient in one or more treatment cycles, wherein each treatment cycle comprises dosing on day 1 and day 8 every two, three, or four weeks, for example every three weeks.

50. The liposome or pharmaceutical composition for use according to any one of claims 26-48, wherein the liposome or pharmaceutical composition is administered to the patient in one or more treatment cycles, wherein each treatment cycle comprises dosing on day 1 and day 8 every three or four weeks.

51. The liposome or pharmaceutical composition for use according to any one of claims 26-50, wherein the liposome or pharmaceutical composition is administered to the patient over from 2 to 8 treatment cycles, such as from 2 to 7 treatment cycles, for example from 3 to 6, such as from 4 to 5 treatment cycles.

52. The liposome or pharmaceutical composition for use according to any one of claims 26-51, wherein the liposome comprises:
a) 1,2-distearoyl-sn-glycero-3-[phospho-rac-(1-glycerol)] (DSPG);
b) 1,2-distearovl-sn-glycero-3-phosphoethanolamine-N-[methoxy (polyethylene glycol)-2000)] (DSPE-PEG);
c) 1,2-distearoyl-sn-glycero-3-phosphocholine (DSPC);
at a mol ratio of: 25:5:70 of a), b) to c),
d) less than 1% cholesterol, and
e) cisplatin.

53. The liposome or pharmaceutical composition for use according to any one of claims 26-52, wherein the use provides for from 80 mg to 120 mg cisplatin per treatment cycle, for example 75 mg.

54. The liposome or pharmaceutical composition for use according to any one of claims 26-53, wherein each dose of the anti-cancer agent administered to the patient is from 20 to 60 mg/m², such as from 30 to 50 mg/m², for example 40 mg/m².

55. The liposome or pharmaceutical composition for use according to claim 54, wherein the anti-cancer agent is cisplatin or carboplatin, such as cisplatin.

56. The liposome or pharmaceutical composition for use according to any one of the preceding claims, wherein the liposome or pharmaceutical composition is administered to the patient in combination with one or more further anti-cancer agents.

57. A liposome for use in the treatment of breast cancer, such as metastatic breast cancer in a patient having a DRP of at least 80%, comprising administering the liposome of claim 20 to the patient.
